(19) United States
(12) Patent Application Publication
(10) Pub. No.: US 2023/0137633 A1
(43) Pub. Date: May 4, 2023

(54) EXPANDABLE INTRODUCER SHEATH FOR MEDICAL DEVICE

(71) Applicant: ABIOMED, Inc., Danvers, MA (US)

(72) Inventors: Christopher Nason Korkuch, Danvers, MA (US); Jonathan Barry, Danvers, MA (US); Anne Gabrielle McLoughlin, Danvers, MA (US); Robert Kirkpatrick, Danvers, MA (US); Ying Xu, Danvers, MA (US); Glen R. Fantuzzi, Danvers, MA (US); Robert Fishman, Danvers, MA (US); Mithun Rajaram, Danvers, MA (US); Matthew D'Agostino, Danvers, MA (US)

(73) Assignee: ABIOMED, Inc., Danvers, MA (US)

(21) Appl. No.: 17/974,131

(22) Filed: Oct. 26, 2022

Related U.S. Application Data
(60) Provisional application No. 63/272,951, filed on Oct. 28, 2021.

(51) Int. Cl. A61F 2/915

(52) U.S. Cl. CPC A61F 2/915; A61F 2002/9155

(57) ABSTRACT

Expandable introducer sheaths and associated laser cut frames for the insertion of a medical device into a blood vessel. In some examples, an expandable sheath may have a frame including a plurality of radial expansion bands and a plurality of connecting bridges for connecting adjacent radial expansion bands. The radial expansion bands are configured to accommodate radial expansion and the plurality of connecting bridges are configured to be longitudinally expandable and to impart column strength as a medical device (e.g., an intracardiac heart pump) is passed through the sheath, such as during insertion or removal of the medical device.

### BACKGROUND OF THE INVENTION

Intracardiac heart pump assemblies can be introduced into the heart either surgically or percutaneously and used to deliver blood from one location in the heart or circulatory system to another location in the heart or circulatory system. For example, when deployed in the heart, an intracardiac pump can pump blood from the left ventricle of the heart into the aorta, or pump blood from the inferior vena cava into the pulmonary artery. Intracardiac pumps can be powered by a motor located outside of the patient’s body (and accompanying drive cable) or by an onboard motor located inside the patient’s body. Some intracardiac blood pump systems can operate in parallel with the native heart to supplement cardiac output and partially or fully unload components of the heart. Examples of such systems include the IMPELLA® family of devices (Abiomed, Inc., Danvers MA).

In one common approach, an intracardiac blood pump is inserted by a catheterization procedure through the femoral artery using a sheath, such as a peel away introducer sheath. The sheath can alternatively be inserted in other locations such as in the femoral vein or any path for delivery of a pump for supporting either the left or right side of the heart.

The introducer sheath can be inserted into the femoral artery through an arteriotomy to create an insertion path for the pump assembly. A portion of the pump assembly is then advanced through an inner lumen of the introducer and into the artery. Once the pump assembly has been inserted, the introducer sheath is peeled away. A repositioning sheath can then be advanced over the pump assembly and into the arteriotomy. Replacing the introducer sheath with the repositioning sheath during insertion of a medical device can reduce limb ischemia and bleeding at the insertion site in the skin (and/or at the insertion site within the vessel) because of better fixation of the sheath to the patient when used with a hemostatic valve.

Since commercially available tear away introducer sheaths are not radially expandable, the inner diameter of the introducer sheath must always be large enough to accommodate the largest diameter portion of the pump assembly such as the pump head even if other parts of the pump assembly, such as the catheter, have a significantly smaller diameter. In this example, the introducer creates an opening that has an outer diameter wider than necessary to allow passage of the pump catheter into the vessel. Then, the introducer sheath is peeled or torn away and replaced with a lower-profile repositioning sheath. Removing the introducer sheath by peeling it away presents several challenges. For example, introducers can tear too easily and/or prematurely, leading to bleeding or vascular complications. Some introducers may require excessive force to tear away for removal. If a physician applies too much force, when the introducer finally tears, the physician may inadvertently shift the position of the pump within the heart. This configuration also complicates the design of the hemostatic valve located in the hub of the introducer which also needs to tear. Further, a peel away introducer sheath leads to a larger vessel opening after the system is removed, which can complicate vessel closure.

Medical introducers for other applications than inserting heart pumps have expandable sheath bodies which may expand radially to allow passage of percutaneous devices into the patient’s vasculature. These existing expandable introducers are for relatively short-term use and may be designed to prevent thrombosis between the sheath body and an indwelling catheter. These introducers are inserted having inner diameters smaller than the outer diameter of the device being introduced. The introducers expand to allow passage of the device through the sheath and into the vasculature and then may shrink again after the device has passed. In some cases, these expandable introducers require a distinct expandable feature, e.g., a longitudinal fold or crease or a lumen for injection of a fluid (e.g., saline) to transition from a compressed state to an expanded state. Because these existing expandable introducers are intended for relatively short-term use, clot formation on the outside of the introducer sheath may be unlikely. However, if left in for longer periods of time (e.g., >1 hour, >2 hours, >6 hours, >1 day, >2 days, >1 week), clots may form on the outside surface of the expandable sheath mesh, and risk being dislodged into the blood stream at a later time. Additionally, some commercially available expandable sheaths are completely flexible and therefore do not provide any rigidity within their structure thereby leading to kinking or buckling during insertion or withdrawal of a percutaneous medical device.

### SUMMARY OF THE INVENTION

Systems, devices and methods for insertion of a medical device (e.g., intravascular medical device) are presented. The devices are delivered through an expandable introducer sheath. The expandable introducer sheath is configured to remain in an insertion path (e.g., an arteriotomy) for relatively long durations (e.g., > 1 hr, >2 hr, >6 hr, or any suitable duration). Use of an introducer sheath capable of expansion allows a smaller size sheath to be used for insertion and can allow the vessel opening to spend less time at a larger diameter, notwithstanding the sheath being used for longer durations. For example, the expandable introducer sheath can more easily recoil to a smaller diameter after insertion of the pump, which allows the opening of the vessel to recoil to a more natural position. Additionally, because the medical device only momentarily passes through the vessel wall, the opening in the vessel is expected to be smaller than if a larger non-expandable sheath is used. Still further, since the medical device only momentarily passes through the vessel, friction between the device, sheath, and vessel wall is minimized and there is a reduced axial load and reduced stress on the vessel. That is, the sheath is a smaller size and is therefore not pushing or pulling the vessel along the axis of the insertion/removal path. Instead, when the device passes through the vessel, the vessel is expanded outward radially.

An expandable introducer sheath structure comprises at least one frame and one coating. A coating is applied to the surface of the sheath to facilitate passage inside the patient. Optionally, and in some structures, the coating is applied on the inner surface of the sheath, which is an inner diameter biased approach. An inner-diameter biased coating advantageously provides for a thin coating thickness and, advantageously a relatively smaller force is required to expand the sheath compared to a force required to expand a sheath having a coating without any bias. In alternative embodiments, the coating is applied on the outer surface of the sheath, which is an outer diameter biased approach. An outer-diameter biased coating advantageously provides a smooth outer surface which reduces the risk of clot formation and minimizes friction when inserting a device through the expandable sheath. For example, the use of a smooth outer surface advantageously minimizes the risk of clots forming on the surface of the expandable sheath, and a corrugated inner surface minimizes the surface area of the expandable sheath in contact with a device being pushed through, thereby minimizing associated friction forces. The outer-diameter biased coating further advantageously provides for a thin coating thickness, and advantageously a relatively smaller force is required to expand the sheath compared to a force required to expand a sheath having a coating without any bias. The outer-diameter biased coating advantageously allows the sheath frame to expand and contract as desired, i.e., the outer-diameter biased coating does not immobilize the frame at a fixed diameter because the thin coating thickness is such that the coating does not encapsulate the portions of the frame where frame elements intersect.

The expandable sheath may be configured for insertion into the vasculature of a patient with a dilator assembly.

The expandable introducer sheath structure can be manufactured using thermal bonding or an outer-diameter biased dipping. Advantageously, thermal bonding or an outer-diameter biased dipping produce the smooth outer surface of the sheath, without losing the desired spring-like expandable nature of the sheath.

Since the expandable introducer sheath need not be removed and replaced by a secondary repositioning sheath, the risk of premature tearing/peeling is essentially eliminated and the risk of shifting the introduced device inadvertently (e.g., by overuse of force) is reduced or eliminated. Furthermore, allowing the expandable introducer sheath to remain in an insertion path simplifies the process of inserting the introduced device by reducing the number of steps in the insertion procedure, e.g., by eliminating a second step where the sheath and valve must be peeled away or torn before it is removed.

Such an expandable sheath also does away with the need for the conventional set up of having multiple sheaths, such as a peel away introducer sheath and a repositioning sheath for the introduction of a medical device (e.g., an intracardiac heart pump) into the vessel opening (e.g., arteriotomy). Such an expandable sheath allows a repositioning sheath to be used in conjunction with it, if necessary, but does not require one in all cases. Once the expandable sheath is positioned, it maintains access to a vessel even after the medical device is removed, should such access be required for other medical procedures. This increases procedural efficiency of any medical procedure as there is no need to peel away the introducer sheath for the insertion of a repositioning sheath each time access to the vessel opening is required. Furthermore, more accurate repositioning of the medical device can be achieved with the expandable introducer sheath as the expandable introducer sheath is fixed in position once inserted, whereas the insertion of a separate repositioning sheath involves multiple steps that increase the chances of misplacing the medical device.

The expandable sheath therefore removes the need for multiple sheaths (e.g., an introducer sheath and a repositioning sheath) during any medical procedure requiring access to an opening of a blood vessel of a patient. In particular, the use of a frame and coating assembly which can expand and collapse while being resistant to kinking, and return to its original shape after deformation, advantageously enables delivery and recovery of the medical device. The consolidation of the introducer sheath and the repositioning sheath into a single device can decrease the costs involved during a medical procedure. Further, since only a single sheath is required to gain arteriotomic access to a vessel, less bleeding may be involved during its long-term use with a percutaneous medical device, such as a heart pump. In addition, configuring the expandable sheath for compatibility with a dilator assembly and a stylet assembly reduces issues with dilator insertion and removal as well as improves hemostasis performance. In some cases, a combination of a dual-dilator assembly, an expandable sheath and a hemostasis stylet may provide a synergistic system which can be used relatively early in a procedure, e.g., in a catheterization lab rather than later in procedure, e.g., in surgery, when displacement of the pump could have more severe consequences for a patient. Because such a system can be used relatively early in a procedure, potential pump migration may be addressed earlier, and vascular injury can be reduced.

In some aspects of the technology, the expandable sheath may have a frame extending longitudinally between a proximal end and a distal end of the sheath. The frame is formed by patterning a hypotube having a lumen therein. The hypotube is patterned by laser cutting. The pattern that is introduced into the hypotube is provided to control axial expansion, radial expansion, and compression of the lumen. In some examples, the frame may include a plurality of radial expansion bands and a plurality of connection portions (referred to as “connecting bridges” herein as they bridge the radial expansion bands as described herein) for connecting each of the radial expansion bands, where the plurality of radial expansion bands are configured to be primarily radially expandable and the plurality of connecting bridges are configured to both allow for longitudinal expansion and also provide column strength as a medical device (e.g., an intracardiac heart pump) passed through the sheath (e.g., during insertion or removal of the medical device). Moreover, the longitudinal expansion and column strength provided by the connecting bridges may aid in avoiding kinking when the sheath undergoes bending deformation. As explained in more detail below, in some examples, the radial expansion bands may include a plurality of band segments extending around a circumference of the frame (e.g., substantially perpendicular to a longitudinal axis of the frame or obliquely relative to the longitudinal axis) and the radial expansion bands and connecting bridges may alternate along the length of the frame.

### BRIEF DESCRIPTION OF THE DRAWINGS

The foregoing and other objects and advantages will be apparent upon consideration of the following detailed description, taken in conjunction with the accompanying drawings, in which like reference characters refer to like parts throughout, and in which:

FIG. 1 shows an isometric view of an illustrative sheath assembly including an illustrative expandable sheath coupled to an illustrative dilator assembly;

FIG. 2 shows an isometric view of an illustrative expandable sheath assembly;

FIG. 3 shows an illustrative introducer sheath after insertion into the blood vessel;

FIG. 4 shows an isometric view of an illustrative pump being inserted through an illustrative expandable sheath;

FIG. 5 shows an isometric view of an illustrative pump being removed from an illustrative expandable sheath;

FIG. 6 shows a partial side view of an exemplary laser cut frame design for a sheath body as it may appear when flattened, in accordance with aspects of the disclosure;

FIG. 7 shows a partial side view of the laser cut frame of FIG. 6 as it may appear when rolled;

FIGS. 8-19 show partial side views of various exemplary configurations of laser cut frames based on the frame design of FIG. 7, in accordance with aspects of the disclosure;

FIGS. 20-23 show partial perspective views of various exemplary designs of laser cut frames for a sheath body, in accordance with aspects of the disclosure;

FIGS. 24-27 show partial perspective views of various exemplary designs of laser cut frames for a sheath body, in accordance with aspects of the disclosure;

FIGS. 28-33 show partial perspective views of various exemplary designs of laser cut frames for a sheath body, in accordance with aspects of the disclosure;

FIG. 34 shows a partial perspective view of an exemplary design of a laser cut frame for a sheath body, in accordance with aspects of the disclosure;

FIGS. 35-37 show partial perspective views of various exemplary designs of laser cut frames for a sheath body, in accordance with aspects of the disclosure;

FIG. 38 shows a partial perspective view of an exemplary design of a laser cut frame for a sheath body, in accordance with aspects of the disclosure; and

FIGS. 39-44 show partial perspective views of various exemplary designs of laser cut frames for a sheath body, in accordance with aspects of the disclosure.

### DETAILED DESCRIPTION

To provide an overall understanding of the systems, method, and devices described herein, certain illustrative embodiments will be described. Although the embodiments and features described herein are specifically described for use in connection with an intracardiac heart pump system, it will be understood that all the components and other features outlined below may be combined with one another in any suitable manner and may be adapted and applied to other types of medical devices such as electrophysiology study and catheter ablation devices, angioplasty and stenting devices, angiographic catheters, peripherally inserted central catheters, central venous catheters, midline catheters, peripheral catheters, inferior vena cava filters, abdominal aortic aneurysm therapy devices, thrombectomy devices, TAVR delivery systems, cardiac therapy and cardiac assist devices, including balloon pumps, cardiac assist devices implanted using a surgical incision, and any other venous or arterial based introduced catheters and devices.

The systems, methods and devices described herein may provide an expandable sheath assembly for the insertion of a medical device (e.g., an intracardiac heart pump) into a blood vessel through a vessel aperture. The expandable sheath assembly may comprise a dilator assembly and a sheath body having an inner surface and an outer surface, the inner surface defining a lumen that extends between proximal and distal ends of the sheath. Optionally, the expandable sheath assembly may include a hemostasis stylet. The expandable sheath assemblies (including the sheath body, dilator assembly, and optional hemostasis stylet) may provide advantages over existing expandable sheath assemblies for patients with coronary artery disease (CAD) and peripheral artery disease, presenting with calcification and tortuosity of arteries, making delivery of introducer sheaths and catheters difficult. The expandable sheath assemblies herein may also be easier to insert than traditional assemblies because of their reduced insertion profile, increased flexibility, reduced friction, reduced risk of kinking under bending, and increased column strength under compression. The reduced insertion profile may minimize insertion related complications, may minimize stretching and load on the vessel opening, and may minimize the risk of limb ischemia. The structure of the sheath body described herein may provide sufficient axial stiffness for pushability and buckling resistance, while maintaining bending flexibility and kink resistance, and may further decouple axial extension and radial compression to reduce or prevent “finger trapping.” Moreover, the structures of the sheath body described herein may provide an improvement over existing introducer sheaths bodies by either, having a smooth inner surface with a thin coating thickness reducing the force required to expand the sheath compared to the force required to expand the sheath having a coating without any bias, or having a smooth outer surface reducing the risk of thrombus formation during use over longer durations while at the same time enabling the sheath to expand and contract as desired and reducing friction between the sheath body and devices being inserted through it.

Moreover, the momentary expansion of the sheath body may minimize the size of the opening, e.g., arteriotomy, required when inserting the sheath into the vasculature of the patient. Minimizing the amount of time, the sheath body is in an expanded state may also minimize damage to a vessel wall as a smaller opening would be required to accommodate the sheath body in the relaxed or collapsed state, thereby minimizing thrombotic occlusion of the vessel. A smaller opening may also minimize the time to reach hemostasis after removal of the medical device. Such an expandable sheath may reduce or eliminate a need for having multiple sheaths, such as a peel away introducer sheath and a repositioning sheath for the introduction of amedical device (e.g., an intracardiac heart pump) into the vessel. Nevertheless, such an expandable sheath may also be used with multiple sheaths where desired. Once the expandable sheath is positioned in an opening of a blood vessel of a patient, it may maintain access to the vessel even after the medical device is removed, should such access be required for other medical procedures. This may increase procedural efficiency of any medical procedure by reducing or eliminating a need to re-gain alternative access or re-insert a second sheath in the same access site. The effective consolidation of the introducer sheath and the repositioning sheath into a single device may decrease the costs involved during a medical procedure. Further, since only a single sheath may be required to gain arteriotomic access to a vessel, less bleeding may be involved during long term use of a percutaneous medical device such as a heart pump. The integration of the sheath body and dilator assembly with a hemostasis stylet may further allow for titrated hemostasis at the vessel opening. In some implementations, the hemostasis stylet can be a repositioning sheath, which may also be used to control blood flow along the expandable sheath and minimize bleeding.

Additionally, the expandable sheath assemblies herein may be used to maintain guidewire access throughout a full medical procedure, thus allowing a user to remove a medical device (e.g., heart pump) while the expandable sheath assembly remains in place.

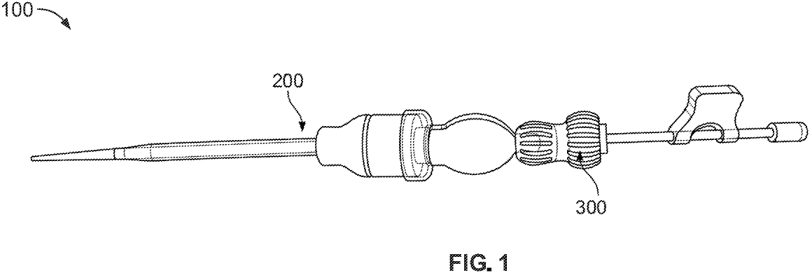

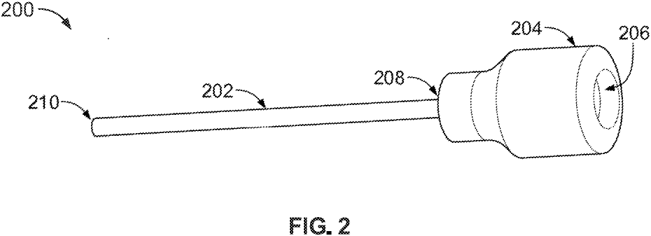

FIGS. 1-6 show different aspects of an illustrative sheath assembly, and exemplary components and configurations. FIG. 1 shows an illustrative sheath assembly including an expandable introducer sheath 200 (further described in relation to FIG. 2) coupled to a dilator assembly 300. As will be understood, the expandable introducer sheath 200 may be attached to the dilator assembly 300 prior to insertion into a desired location in blood vessel. After the expandable introducer sheath 200 and dilator assembly 300 are at the desired location in the blood vessel, the dilator assembly may be removed from the blood vessel, and a medical device, e.g., a pump, is introduced through the expandable introducer sheath 200.

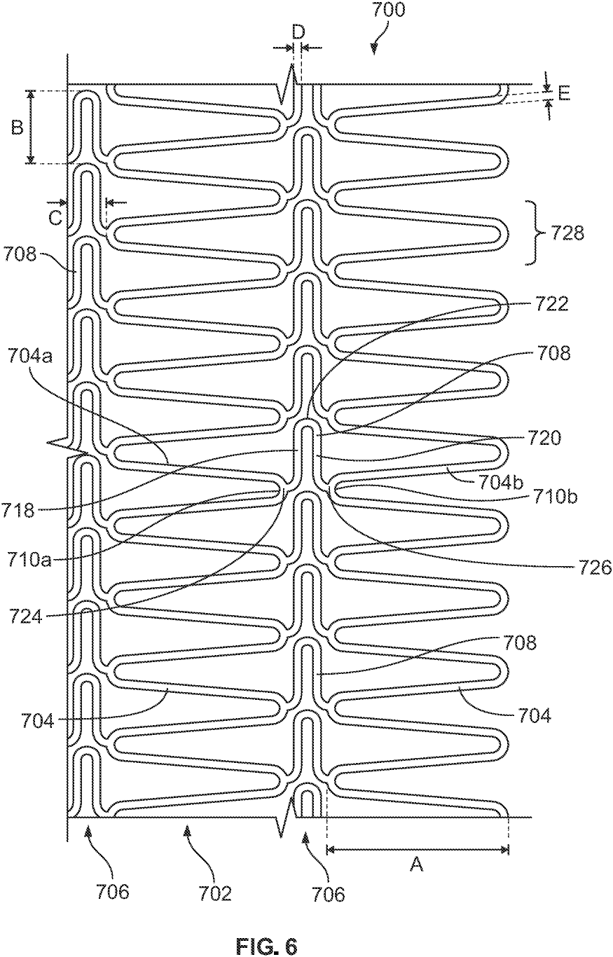

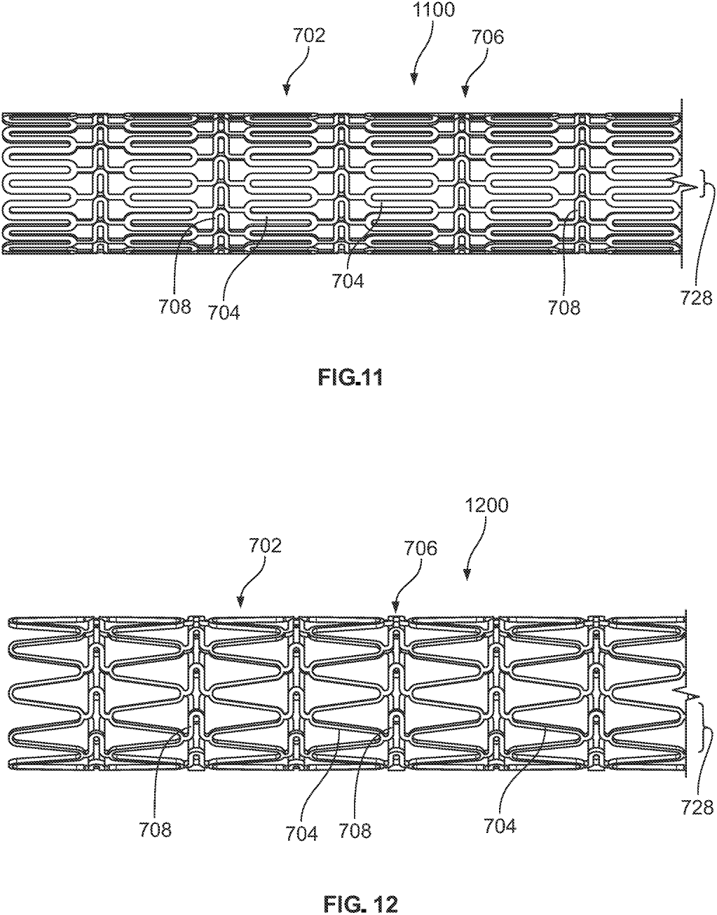

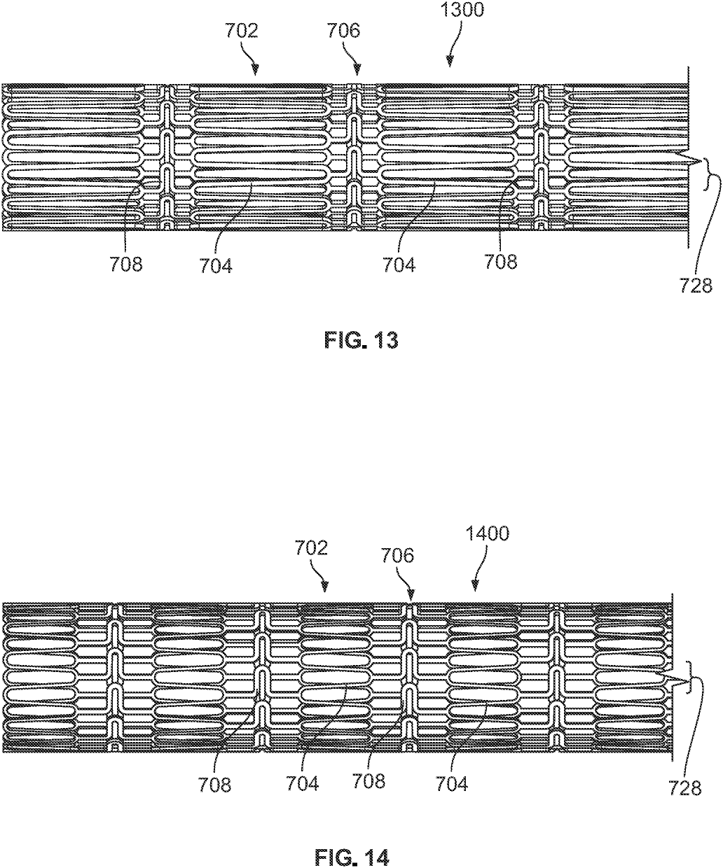

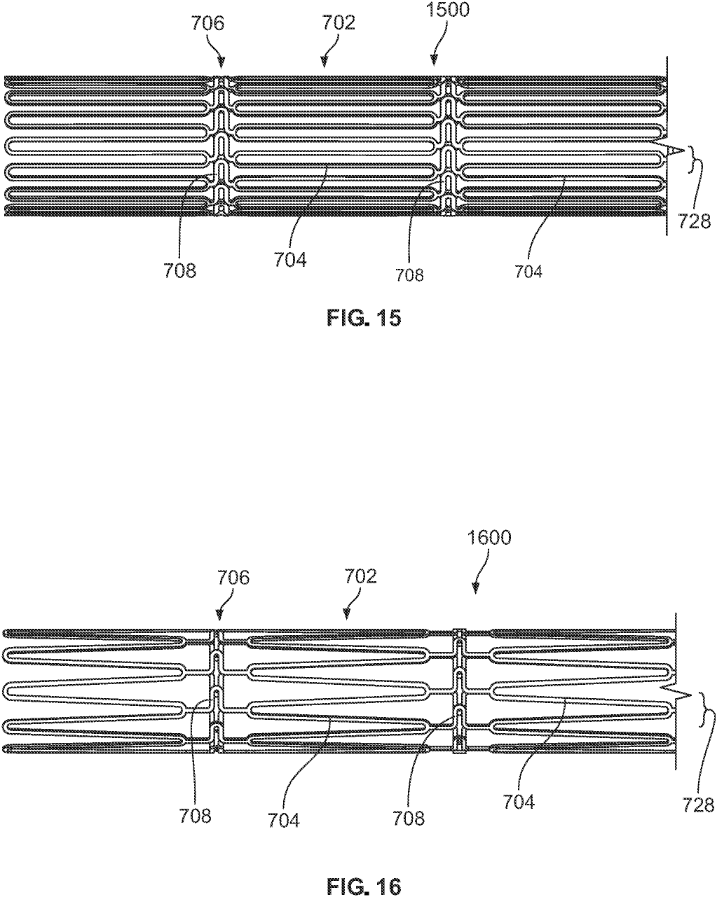

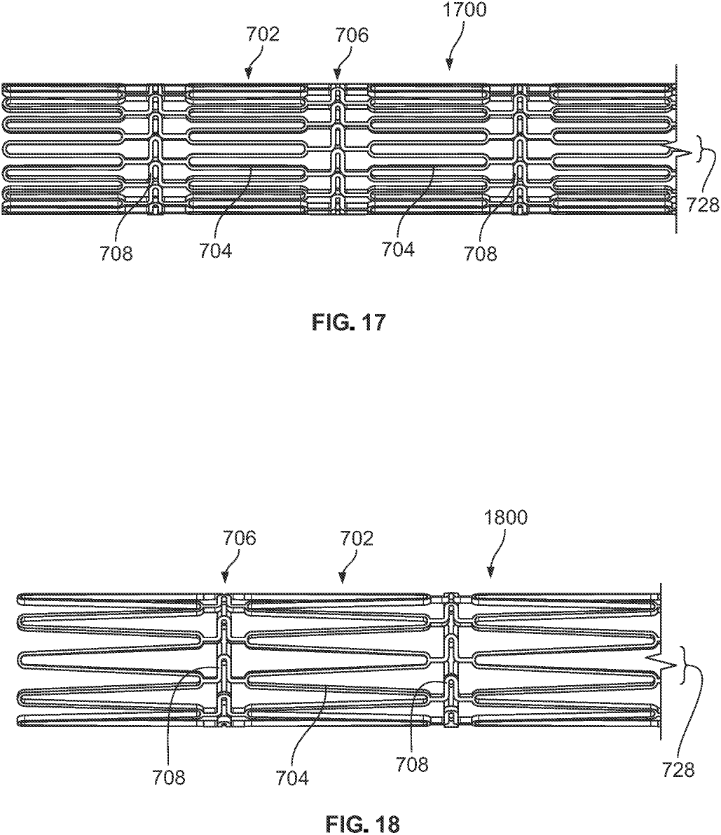

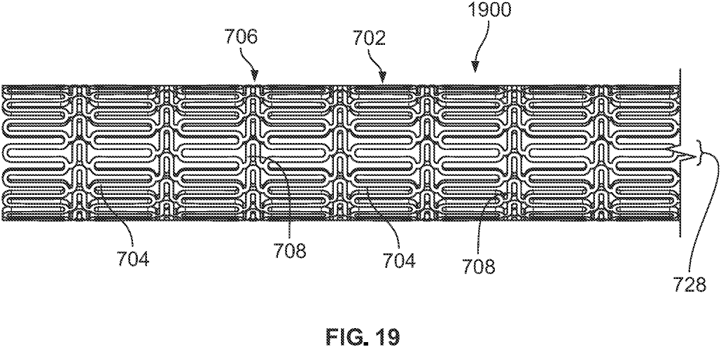

FIG. 2 shows an illustrative expandable introducer sheath 200 (e.g., the expandable introducer sheath 200 of FIG. 1), comprising a hub 204 and expandable sheath body 202. As discussed further below in relation to FIGS. 6-38, the expandable sheath body 202 of the expandable introducer sheath 200 may comprise a frame and one or more coatings. In one embodiment, the expandable sheath body 202 may include a frame (e.g., such as those described below with respect to FIGS. 6-38), a coating encapsulating the frame (e.g., a polymer, elastomer, etc.). In addition, in some cases, the expandable sheath body 202 may further include a hydrophilic coating on a portion of an inner surface and/or outer surface of the coated frame. The frame of the expandable sheath body 202 shown in FIG. 2 may be a laser cut frame made of any suitable metal (e.g., Nitinol, stainless steel) or polymer (e.g., PEEK). Exemplary laser cut frame designs are further described below. In some cases, the frame of the expandable sheath body 202 may be configured such that the force to insert a device within the sheath body is minimized (e.g., below 5 N), and such that the sheath can turn corners as required by patient anatomy (e.g., turn corners with a minimum bend configuration of 30 mm through 55-degree bend using a force less than 5 N). In some cases, the hydrophilic coating may provide the advantage of reducing frictional forces on the sheath, which may reduce the likelihood of “finger trapping,” in which a device being inserted (e.g., an intracardiac heart pump) may stretch the sheath axially, causing at least a portion of the sheath’s diameter to compress upon the device being inserted, which may in turn further increase the frictional force on the inserted device. In addition, in some cases, the exemplary laser cut frame designs described herein may be configured to reduce or eliminate the relationship between axial extension and radial compression such that the likelihood of “finger trapping” is reduced or eliminated.

As shown in FIG. 2, the expandable sheath body 202 may have a proximal end 208, a distal end 210, and a lumen extending between the proximal and distal end. On the proximal end 208, the expandable sheath body 202 may be attached to a hub 204. The hub 204 may also have a proximal end and a distal end, with a lumen extending between the proximal end and the distal end. On its distal end, the hub 204 may be attached to the expandable sheath body 202. On the proximal side of the hub 204 there may be a hemostasis valve 206 within the sheath hub 204. The hemostasis valve 206 may be configured to allow for insertion of components through the hub and sheath while preventing the flow of fluid (i.e., blood) from the distal end of the expandable sheath body 202 to the outside of the expandable sheath body 202 and hub 204. In some cases, the hub 204 may further contain a side-arm (not shown in FIG. 2) that allows for aspiration of fluid and flushing of the sheath. Further, the distal end 210 of the expandable sheath body 202 may have a geometry configured to interface with a dilator assembly. The distal end 210 of the expandable sheath body 202 may also be configured to be atraumatic to reduce or eliminate the likelihood of damaging the blood vessel wall or any other anatomy during insertion of the expandable introducer sheath assembly, and while the expandable introducer sheath assembly remains within a patient.

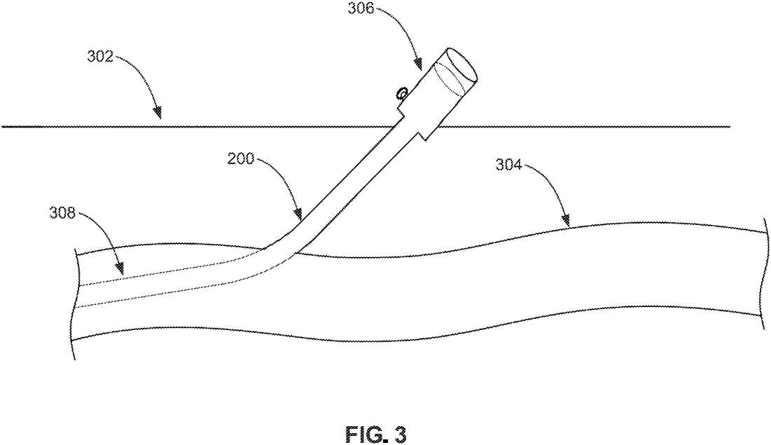

FIG. 3 shows an illustrative expandable introducer sheath 200 (e.g., expandable introducer sheath 200 shown in FIG. 2) after insertion into the blood vessel 304 of a patient. As shown, the proximal portion 306 of the introducer sheath 200 may remain outside the skin 302 of the patient, while the distal portion 308 of the expandable introducer sheath 200 is within the vessel 304. As already described, some or all of the expandable introducer sheath 200 may be coated with one or more coatings. Thus, in some examples, at least a distal portion (e.g., portion 308) of may be coated with one or more coatings. Likewise, in some examples, the entire length of the expandable introducer sheath 200 may be coated with one or more coatings. In some cases, a coating of the expandable introducer sheath 200 may be configured to minimize friction between various materials of the sheath and the device being inserted. Likewise, in some cases, a coating of the expandable introducer sheath 200 may be configured to seal all voids in the sheath’s frame to form a closed-cell mesh. Forming a close-celled mesh such that the expandable sheath is not porous may prevent blood or flushing liquids from seeping through the expandable sheath. In addition, forming a closed-cell mesh may allow the expandable introducer sheath 200 to be less sensitive to positioning relative to the arteriotomy because the closed-cell mesh reduces risk of bleeding across the arteriotomy. As described further below, expandable sheath bodies using the frame designs described herein may provide sufficient stiffness for the sheath body to maintain an open lumen along its length, while remaining flexible enough to expand. In addition, in instances where a medical device being passed through the sheath has a feature that tends to compress the expandable sheath body in an axial direction while the device is being withdrawn (e.g., a bulbous pump head or pigtail at a distal end of an intracardiac heart pump), the expandable sheath bodies described herein may be configured to provide sufficient longitudinal resistance or column strength to counter the removal force and prevent the sheath from buckling and/or bunching up along its length during removal of the device.

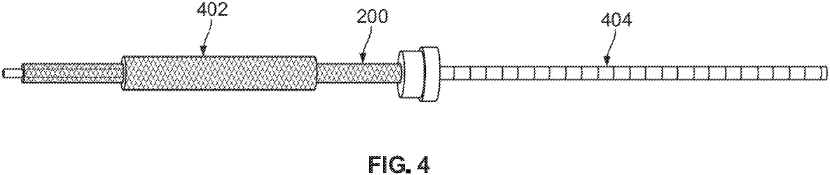

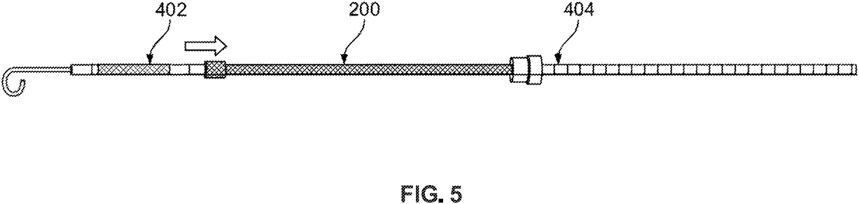

As an example, FIG. 4 shows the insertion of a heart pump 402 through an introducer sheath 200. As shown in the example of FIG. 4, the outer diameter of the pump 402 is larger than the inner diameter of the expandable sheath body 202 causing the expandable sheath body 202 to radially expand as the pump passes through. The outer diameter of catheter 404 may be larger than the inner diameter of the expandable sheath body 202, where the expandable sheath body 202 collapses back down on the catheter 404 after passage of the pump 402 and the expandable sheath body 202 tight on the catheter 404. FIG. 5 is an exemplary illustration of the removal of the pump 402. The distal end of the introducer sheath 200 may expand radially as the pump 402 is pulled through. In addition, the column strength of the introducer sheath 200 may determine whether the introducer sheath 200 will resist buckling due to the frictional loads of the pump 402 and of the uncurl force of the pigtail.

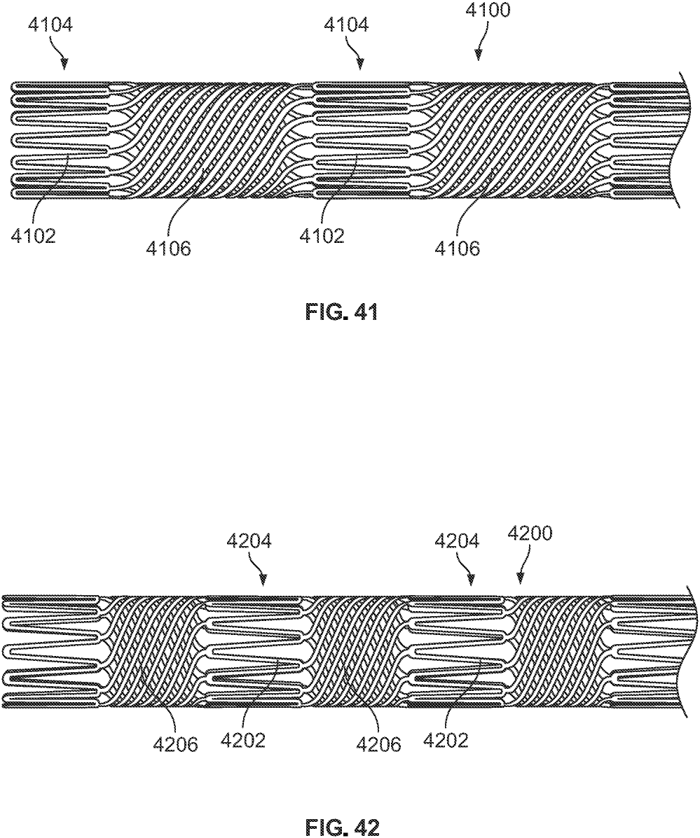

As mentioned above, the frame component of the expandable sheath body may be a laser cut frame (e.g., FIGS. 6-44). The laser cut frame may be made from any suitable material, such as Nitinol, stainless steel, or another suitable metal. Likewise, in some examples the frame material may be made from a suitable polymer such as PEEK. At least one advantage of using Nitinol for the frame is that the frame may be visible under medical imaging, e.g., fluoroscopy, and may thus assist a user with placement of the device. Likewise, in some examples, the frame of expandable sheath body 202 may be radioactive. A further potential advantage of using superelastic Nitinol for the frame is that it may provide increased resistance to kinking and/or mechanical deformation.

FIGS. 6-44 show various illustrative laser cut frame designs for an expandable sheath body in accordance with aspects of the technology.

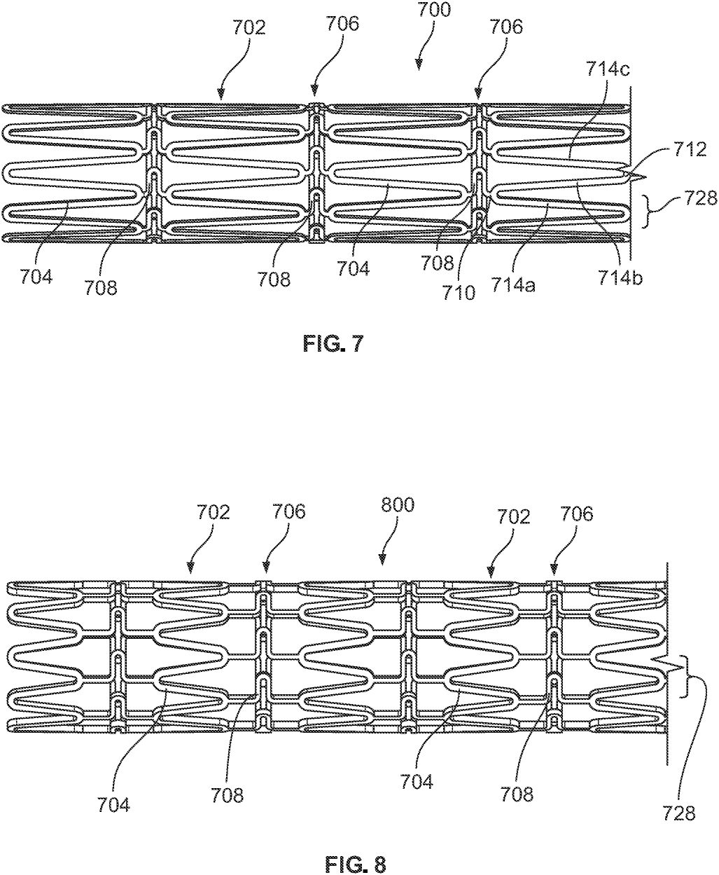

FIG. 6 shows a portion a laser cut frame of a sheath body lumen as it may appear when cut and flattened, and FIG. 7 shows a side-view portion of the sheath body lumen of FIG. 6 as it may appear as it would be incorporated into a sheath (i.e., in a tubular configuration). As shown in FIG. 7, the laser cut frame 700 may include a plurality of radial expansion bands 702 that extend around the circumference of the laser cut frame 700, with each radial expansion band 702 having a band strut 704 arranged in a zigzag pattern, and a plurality of connecting bridges 706 with each connecting bridge 706 having a bridge strut 708. The plurality of radial expansion bands 702 and the plurality of connecting bridges 706 are positioned and arranged in an alternating pattern throughout the entire length of the frame 700.

As illustrated, the plurality of radial expansion bands 702 are configured to allow the frame to expand and contract radially and the plurality of connecting bridges 706 are configured to allow the frame to expand and contract longitudinally in a controlled manner. In some aspects of the technology, the plurality of radial expansion bands 702 may be configured to provide adequate radial expansion to allow a given medical device (e.g., an intracardiac heart pump) to be inserted into and passed through the frame 700, while keeping a ratio of radial expansion to longitudinal contraction low enough to avoid the problem known as finger-trapping. Finger-trapping is used to describe the situation where the longitudinal expansion is such that the frame radially contracts to an extreme, thereby “trapping” any article disposed within the frame. In addition, the plurality of connecting bridges 706 may be configured to enable adequate longitudinal extension to further reduce the chances of finger-trapping when a medical device is inserted through the frame 700, and to lower the chances of kinking when the sheath body is subjected to bending. Further, legs 718, 720 of each bridge strut 708 may be configured to interfere with (i.e., contact) each other after the frame 700 is compressed longitudinally, such that the frame 700 may have a column strength (or longitudinal resistance) sufficient to enable the sheath body to avoid buckling when being pushed into a patient’s vasculature.

As shown in FIGS. 6 and 7, each of the plurality of radial expansion bands 702 may include a plurality of band segments 728 that repeat around the circumference of the frame 700 and in which each band segment forms a zigzag arrangement having a sinusoidal, undulating, or wave-like pattern, in which each band strut 704 has a plurality of first bent end portions 710, a plurality of second bent end portions 712, and a plurality of straight portions 714 connecting the first and second bent end portions 710, 712. For example, as shown in FIG. 7, a straight portion 714a is joined to an adjacent straight portion 714b by a first bent end portion 710, and to a second adjacent straight portion 714c by a second bent end portion 712. According to this aspect, each band segment 728 may span adjacent first bent end portions 710, and includes a pair of straight portions 714 connected by a second bent end portion 712. However, it should be understood that the band segments 728 may be defined by any suitable group of repeating elements formed by the band strut 704. As shown in FIG. 7, the zigzag arrangement formed by the band segments 728 repeats around the entire circumference of the frame 700. In this example, each radial expansion band 702 is formed perpendicular to a longitudinal axis of the frame 700, though it should be appreciated that other configurations are also contemplated. For example, in some examples, the band segments may extend around the circumference obliquely such that the expansion bands 702 are angled with respect to the longitudinal axis of the frame 700. As shown in FIG. 6, each radial expansion band 702 may have a width A which is a longitudinal distance between opposing bent end portions (i.e., between bent portion 710 and bent portions 712), and each band strut 704 may have a thickness E.

As stated above, each connecting bridge 706 is arranged between a bent end portion of a first radial expansion band 702 and a bent end portion of a second radial expansion band 702. As shown in FIG. 6, each connecting bridge 706 may be U-shaped or V-shaped and its respective bridge strut 708 may include a first leg 718, a second leg 720, and a bend 722 connecting the first and second legs 718, 720. Each of the first and second legs 718, 720 may be straight or substantially straight, and may further include an end portion 724, 726 that extends away from the leg to connect to an adjacent bent end portion of one of the band struts 704. For example, in FIG. 6, the end portion 724 of the first leg 718 is shown connecting to the bent end portion 710a of the band strut 704a, and the end portion 726 of the second leg 720 is shown connecting to the bent end portion 710b of the band strut 704b. Also, the connecting bridge structures repeat around the circumference of the frame as illustrated in FIGS. 6-27, and 38. As shown in FIG. 6, each connecting bridge 706 may have a length B and width C, and each bridge strut 708 may have a thickness D. The values of B, C, and D may be varied (e.g., alone, or in combination with other values, such as band width A and band strut thickness E) to provide a frame 700 and sheath body that allows for a desired amount of longitudinal extension under anticipated loads, a desired amount of compressibility prior to the legs (718, 720) interfering with each other (i.e., before they “stack” against each other), a desired amount of flexibility, etc. These bent end portions 710a, 710b are also referred to as “peaks” or “troughs” herein to as they form peaks and troughs in the zigzag pattern that is formed by a series of such bands formed between connecting bridges.

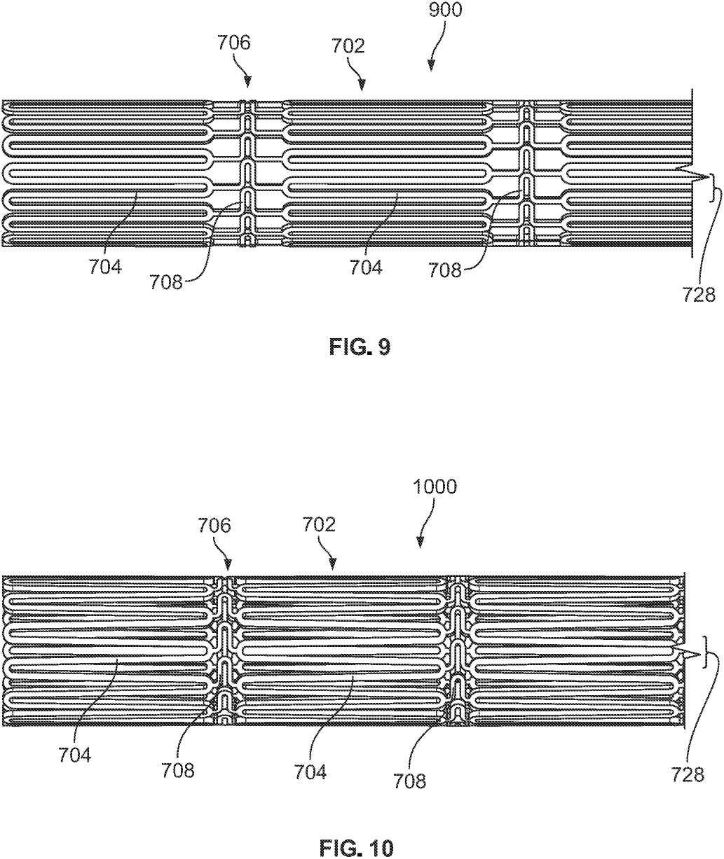

FIGS. 8-19 illustrate various alternate configurations of laser cut frames based on the frame design of FIG. 7. The reference numbers for the features described in FIGS. 6 and 7 are used to designate corresponding features in FIGS. 8-19 to clearly understand the variations in the features among the alternate configurations. As can be seen from FIGS. 8-19, the exemplary frames 800, 900, 1000, 1100, 1200, 1300, 1400, 1500, 1600, 1700, 1800, 1900 each include radial expansion bands 702 with band struts 704 arranged in a zigzag pattern, and connecting bridges 706 with bridge struts 708 that function similarly to those shown in FIG. 7, but with different shapes and relative dimensions that the features described in FIG. 7. For example, the straight portion of each band struts 704 (e.g., straight portions 714a, 714b, 714c of FIG. 7) may be non-parallel (i.e., angled) relative to one another when the frame is in a relaxed state (e.g., as shown in FIGS. 7, 8, 10, 12, 13, 14, 16, and 18) or they may be substantially parallel to each other (e.g., as shown in FIGS. 9, 11, 15, 17, and 19). “Substantially parallel” as used herein, describes the appearance of being parallel instead of the appearance of being non-parallel (i.e., such that, if the struts were extended, they would not intersect at some point). In addition, as can be seen from the figures, the number of zigzag repeats in each radial expansion band 702 may vary (i.e., the number of band segments 728 in the radial expansion bands may vary). For example, FIGS. 7, 8, 12, 16, and 18 show radial expansion bands 702 having zigzag patterns that repeat ten times (e.g., measured peak-to-peak or trough-to-trough), FIGS. 9, 11, 15, 17, and 19 show radial expansion bands 702 having zigzag patterns that repeat sixteen times. Likewise, as can be seen from the figures, the length of the end portions (e.g., end portions 724, 726 of FIG. 6) of each connecting bridge 706 may also vary. For example, the length of each end portion may be relatively shorter as shown in FIGS. 7, 10, 12, 15, and 19, or may be made longer as shown in FIGS. 8, 9, 11, 13, 14, 16, 17, and 18 in order to increase the longitudinal space between the legs (e.g., legs 718, 720 of FIG. 6) of each connecting bridge 706 and the bent end portions (e.g., 710a, 710b of FIG. 6) of each adjacent radial expansion band 702. This length may be adjusted to further control the extent of axial extension or compression in response to radial expansion or contraction of the frame.

Although the examples of FIGS. 6-19 show a variety of different combinations, it is contemplated that laser cut frames according to the present disclosure may include any suitable number of zigzags in each radial expansion band 702 and any suitable number of connecting bridges 706 in each radial expansion band 702. As will be appreciated, these and other values may be selected to tailor or control one or more of the radial expandability, radial compressibility, longitudinal extendibility, longitudinal compressibility, column strength, flexibility, kink resistance, buckling resistance, and/or finger-trapping resistance according to the contemplated use of the sheath body. In addition, although the exemplary frames depicted in FIGS. 6-19 may include radial expansion bands 702 in which the substantially straight portion of each band strut 704 (e.g., straight portions 714a, 714b, 714c of FIG. 7) are substantially parallel or angled relative to one another, it will be appreciated that other designs and configurations may be used for the columns and connecting bridges, as deemed suitable for given application factors. For example, in some aspects of the technology, the zigzag pattern of each radial expansion band 702 may be fully curved (e.g., sinusoidal) such that struts 704 have no straight portions at all.

Furthermore, dimensions (e.g., radial expansion band width A, connecting bridge length B, connecting bridge width C, bridge strut thickness D, band strut thickness E) may be varied as deemed suitable with any of the frame designs described above and/or depicted in FIGS. 6-19. For example, in some aspects of the technology, radial expansion band width A may be between 2 and 6 mm, band strut thickness E may be between 0.1 and 0.2 mm, connecting bridge width C may be between 0.66 and 2 mm, and wall thickness may be between 0.08 and 0.2 mm. Likewise, in some aspects of the technology, the number of zigzag repeats (e.g., measured peak-to-peak or trough-to-trough) in each radial expansion band 702 may be between 10 and 16. In this regard, the table below summarizes selected values used with the exemplary laser cut frame configurations shown in FIGS. 7-19.

| Figure | Wall Thickness (in mm) | Connecting Bridge Width (in mm) | Radial Expansion Band Width (in mm) | Zigzag Repeats | Band Strut Thickness (in mm) |
|---|---|---|---|---|---|
| 7 | 0.08 | 0.66 | 4 | 10 | 0.2 |
| 8 | 0.2 | 2 | 2 | 10 | 0.2 |
| 9 | 0.14 | 2 | 6 | 16 | 0.2 |
| 10 | 0.2 | 0.66 | 6 | 13 | 0.2 |
| 11 | 0.08 | 1.33 | 2 | 16 | 0.2 |
| 12 | 0.14 | 0.66 | 2 | 10 | 0.1 |
| 13 | 0.14 | 1.33 | 4 | 13 | 0.15 |
| 14 | 0.08 | 2 | 2 | 13 | 0.1 |
| 15 | 0.08 | 0.66 | 6 | 16 | 0.1 |
| 16 | 0.08 | 2 | 6 | 10 | 0.15 |
| 17 | 0.2 | 2 | 4 | 16 | 0.1 |
| Figure | Wall Thickness (in mm) | Connecting Bridge Width (in mm) | Radial Expansion Band Width (in mm) | Zigzag Repeats | Band Strut Thickness (in mm) |
| 18 | 0.2 | 1.33 | 6 | 10 | 0.1 |
| 19 | 0.2 | 0.66 | 2 | 16 | 0.15 |

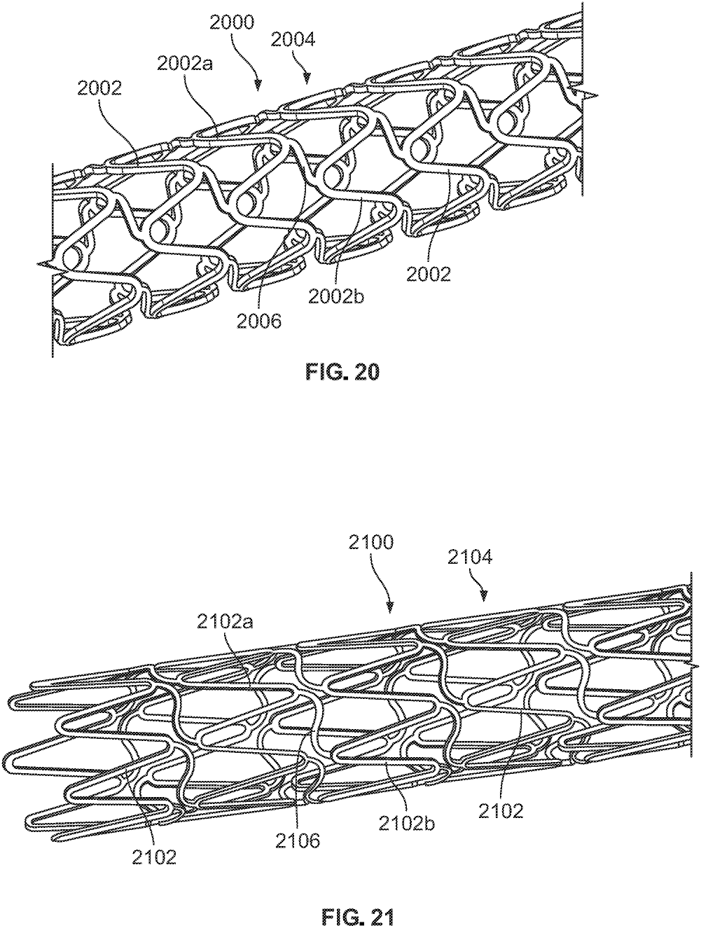

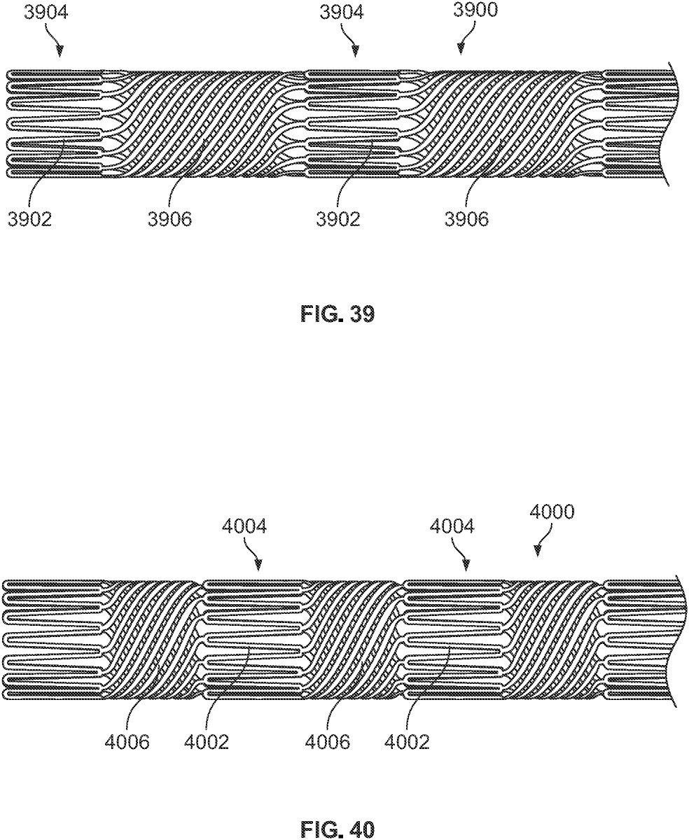

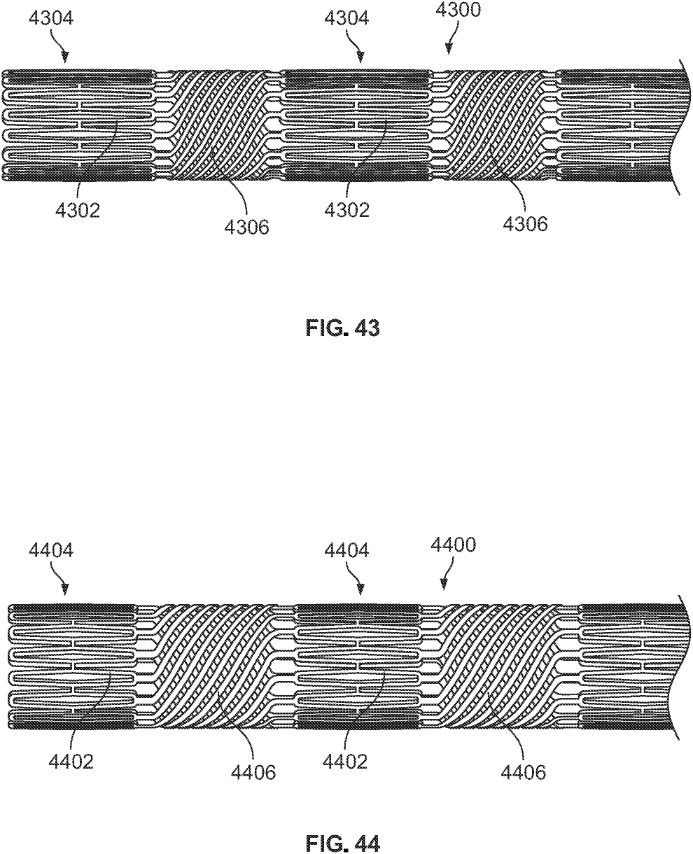

Like FIGS. 7-19, FIGS. 20-23 and 39-42 show exemplary frames 2000, 2100, 2200, 2300, 3900, 4000, 4100, 4200 with radial expansion bands (e.g., 2004, 2104, 2204, 2304, 3904, 4004, 4104, 4204) in which the band struts (e.g., 2002, 2102, 2202, 2302, 3902, 4002, 4102, 4202, respectively) are arranged in a zigzag pattern. FIGS. 43 and 44 show exemplary frames 4300 and 4400 which are similar to those of FIGS. 39-42, but with radial expansion bands 4304, 4404 in which the band struts 4302, 4402 are arranged in a diamond-like or ellipse-like pattern. Unlike FIGS. 7-19, each of the exemplary frame designs of FIGS. 20-23 and 39-44 possess alternative types of structures for the connecting bridges 2006, 2106, 2206, 2306, 3906, 4006, 4106, 4206, 4306, 4406, which that connect to their respective radial expansion bands 2004, 2104, 2204, 2304, 3904, 4004, 4104, 4204, 4304, 4404. For example, the connecting bridges 2006, 2106 are single struts that may be substantially straight struts with curvy ends that connect to the radial expansion band as shown in FIG. 20 or wavy-shaped single struts as shown in FIGS. 21 and 39-44. In FIG. 20, each radial expansion band has peaks and troughs that are out of phase with the adjacent radial expansion band. In FIG. 21, the peak and troughs of each radial expansion band are in phase with the adj acent radial expansion band. As such, each connecting bridge 2006 extends circumferentially from the peak in one radial expansion band to the trough in the adjacent radial expansion band. Contrarily, each connecting bridge 2106 may extend circumferentially to connect between a bent end portion of one band strut in one radial expansion band to a bent end portion of the adjacent band strut in the adjacent radial expansion band (i.e., an out of phase connection instead of the in-phase connections in FIGS. 6-19. For example, in FIG. 20, each radial expansion band 2004 has a zigzag pattern that is in phase with the zigzag pattern of its adjacent radial expansion bands 2004, and each connecting bridge 2006 extends circumferentially from a peak of one zigzag pattern (e.g., that of band strut 2002a) to the nearest trough of the adjacent zigzag pattern (e.g., that of band strut 2002b). FIGS. 39-42 also show an in-phase arrangement for the zigzag pattern of each radial expansion band (3904, 4004, 4104, 4204), but have helical connecting bridges (3906, 4006, 4106, 4206) that extend in both the axial and the circumferential direction such that a peak of a first zigzag pattern in a first radial expansion band will connect to a trough of the adjacent zigzag pattern in the adjacent radial expansion band that is not radially aligned with the peak of the first zigzag pattern in the first radial expansion band. That is, a peak and a trough that are connected by the helical bridge connections are radially offset. On the other hand, in the examples of FIGS. 21, 43, and 44, although each radial expansion band 2104, 4304, 4404 has a pattern that is a mirror image of the pattern of its adjacent radial expansion bands 2004 such that the peaks and troughs of each pattern will naturally approach one another (as is also the case in each of the examples of FIGS. 6-19), each connecting bridge 2106, 4306, 4406 is nevertheless configured to extend circumferentially so that it connects a peak of one pattern (e.g., that of band strut 2102a) to a radially offset trough of the adjacent pattern (e.g., that of band strut 2102b). As will be appreciated, although FIGS. 20-23 and 39-44 each illustrate designs in which each set of radial expansion bands and each set of connecting bridges are positioned in patterns that repeat radially but such radial expansion bands and connecting bridges are laterally static relative to the axis, in some aspects of the technology, a frame may include some radial expansion bands and some connecting bridges that do not repeat radially in a manner that is laterally static relative to the axis (i.e., the radial expansion bands and connecting bridges are not in circular bands with respect to the axis but may instead be oblique with respect to the axis).

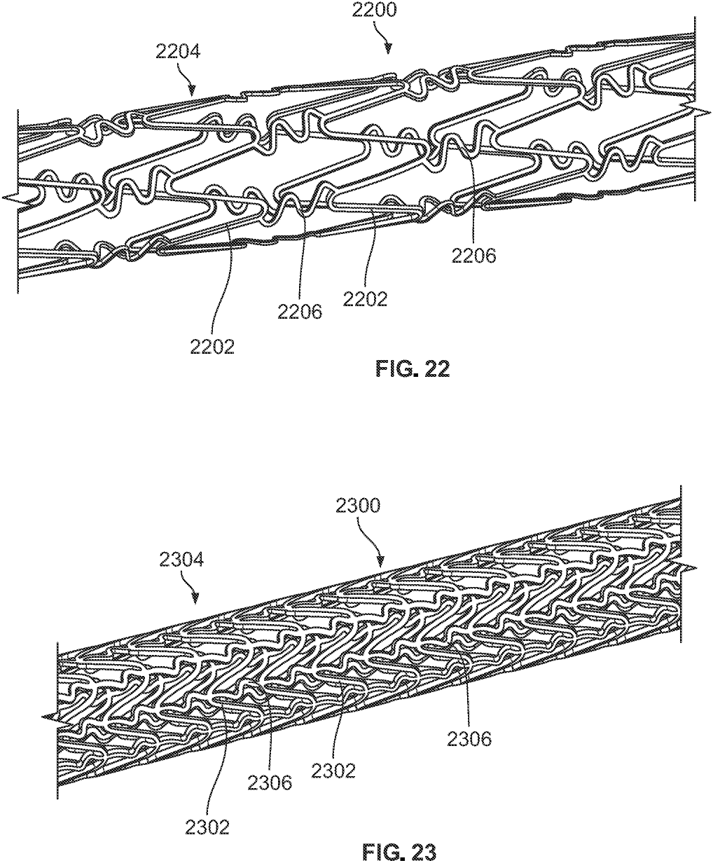

Referring to FIGS. 22 and 23, the connecting bridges 2206, 2306 may have a wavey or zigzag pattern extending in the axial direction. Here as well, the wavey or zigzag-shaped connecting bridges (e.g., 2206, 2306) may be employed where each radial expansion band (e.g., 2204, 2304) has a zigzag pattern (e.g., that of band strut 2202) that is a mirror image of the zigzag pattern of its adjacent radial expansion bands such that the peak of one zigzag pattern will naturally align with the trough of the adj acent pattern as shown in FIG. 22, or where each radial expansion band has a zigzag pattern (e.g., that of band strut 2302) that is aligned with the adjacent radial expansion band such that peaks and troughs are aligned axially as shown in FIG. 23.

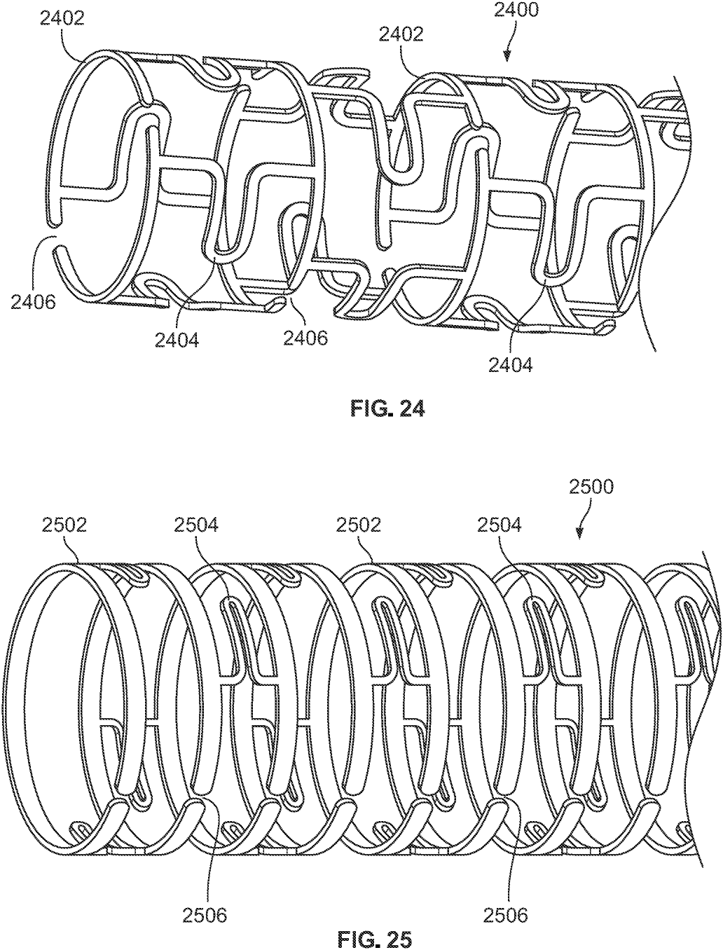

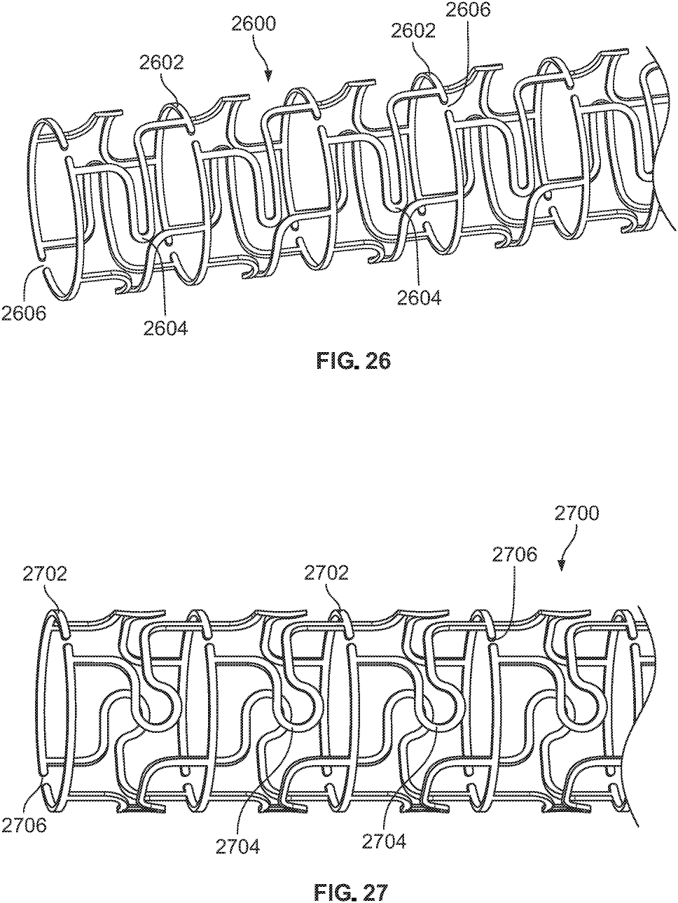

In some examples, a band strut of a radial expansion band need not be continuous around the entirety of a band. For example, several embodiments described below include radial expansion bands formed as split rings including band struts having one or more discontinuous sections coupled to one another by one or more connecting bridges. FIGS. 24-27 show exemplary frames 2400, 2500, 2600, 2700 that each include a series of radial expansion bands formed as split rings 2402, 2502, 2602, 2702 connected by one or more connecting bridges 2404, 2504, 2604, 2704. The connecting bridges may have any suitable shape, such as a U-shaped configuration as shown in FIGS. 24 and 25, an offset U-shaped configuration 2604 as shown in FIG. 26, an offset lobe-shaped configuration 2704 as shown in FIG. 27, etc. In the depicted embodiment, each split ring has one or more gaps 2406, 2506, 2606, 2706 (e.g., one gap as shown FIG. 25, or two gaps as shown in FIGS. 24, 26, and 27). However, in some aspects of the technology, the split ring may include any other suitable number of gaps, such as three, four, five, etc. As shown in FIGS. 24-27, the split rings in each frame may be spaced apart axially by any suitable distance. Further, the axial spacing between each split ring may be uniform along the length of the frame (when the frame is in a relaxed state), or may vary along the length of the frame.

FIG. 28 shows an exemplary frame 2800 that includes a spine 2802 which connects multiple radial expansion bands formed as split rings 2804 that are spaced apart along the length of the frame 2800. In the example of FIG. 28, the spine 2802 is substantially straight in configuration and may be parallel or substantially parallel to a longitudinal axis X of the frame 2800. A plurality of split rings 2804 extend from the spine 2802 in a circumferential direction about longitudinal axis X. Each of the plurality of split rings 2804 may be spaced apart longitudinally by any suitable distance, which may be uniform or varied along the length of the frame.

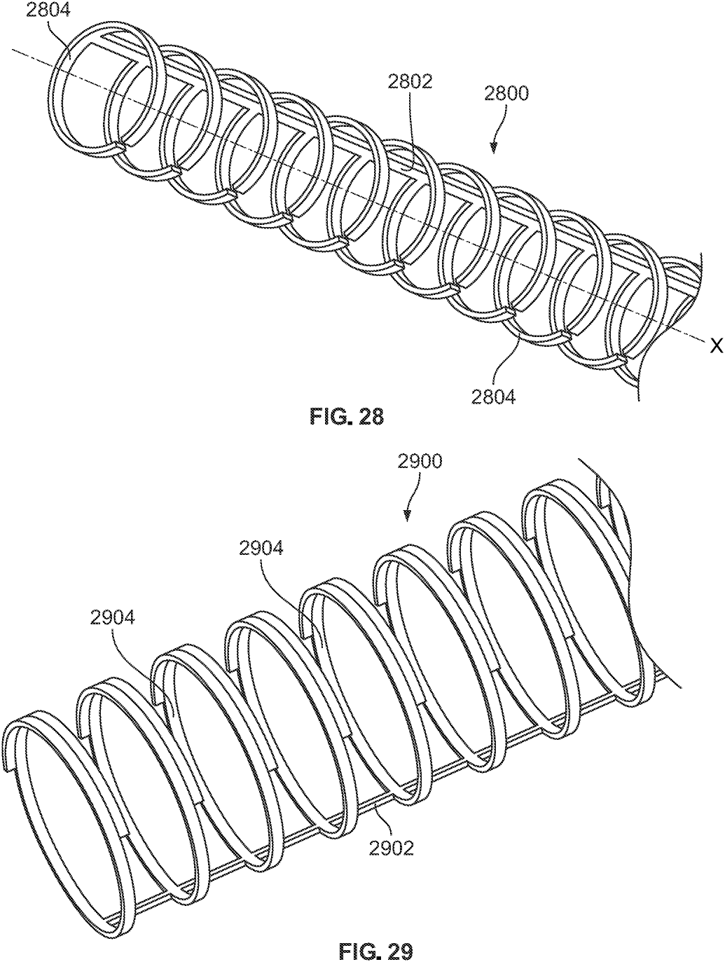

FIG. 29 shows an exemplary frame 2900 similar to frame 2800 of FIG. 28, but which has radial expansion bands formed as split rings with overlapping ends. That is, each of the split rings 2904 have ends that extend past one another when the frame is in a relaxed state. When a device is advance through such a structure, the structure can expand radially but the ends of the rings remain adjacent to control axial expansion. Thus, in the example of FIG. 29, frame 2900 also includes at least one spine 2902 which connects multiple split rings 2904 that are spaced apart along the length of the frame 2900.

FIG. 30 shows an exemplary frame 3000 similar to frame 2800 of FIG. 28, but which includes two spines with two separate sets of split ring radial expansion bands. Thus, in the example of FIG. 30, frame 3000 includes a first spine 3002 and a second spine 3004 which each include multiple split rings that are spaced apart along the length of the frame 3000. In this example, the first and second spines 3002 and 3004 are substantially parallel with each other and are spaced approximately 180 degrees from each other about longitudinal axis X. Likewise, as shown in FIG. 30, each spine 3002, 3004 is substantially straight in configuration and substantially parallel to the longitudinal axis X of the frame 3000. Similar to the frame shown in FIG. 28, a plurality of first split rings 3006 extend from the first spine 3002 in a circumferential direction about the longitudinal axis X, and a plurality of second split rings 3008 extend from the second spine 3004 in a circumferential direction about the longitudinal axis X. Each of the first split rings 3006 terminates before reaching the second spine 3004, and each of the second split rings 3008 terminates before reaching the first spine 3002.

In this example, the plurality of first and second split rings 3006, 3008 are arranged in an alternating pattern along the longitudinal axis, such that each first split ring 3006 is followed by a second split ring 3008. Any other suitable alternating pattern may also be used in this regard. Thus, for example, in some aspects of the technology, 2 first split rings may be followed by 2 second split rings. Likewise, in some aspects of the technology, 2 first split rings may be followed by 1 second split ring. Here as well, the plurality of first split rings 3006 and the plurality of second split rings 3008 may each be spaced apart longitudinally by any suitable distance, which may be uniform or varied along the length of the frame.

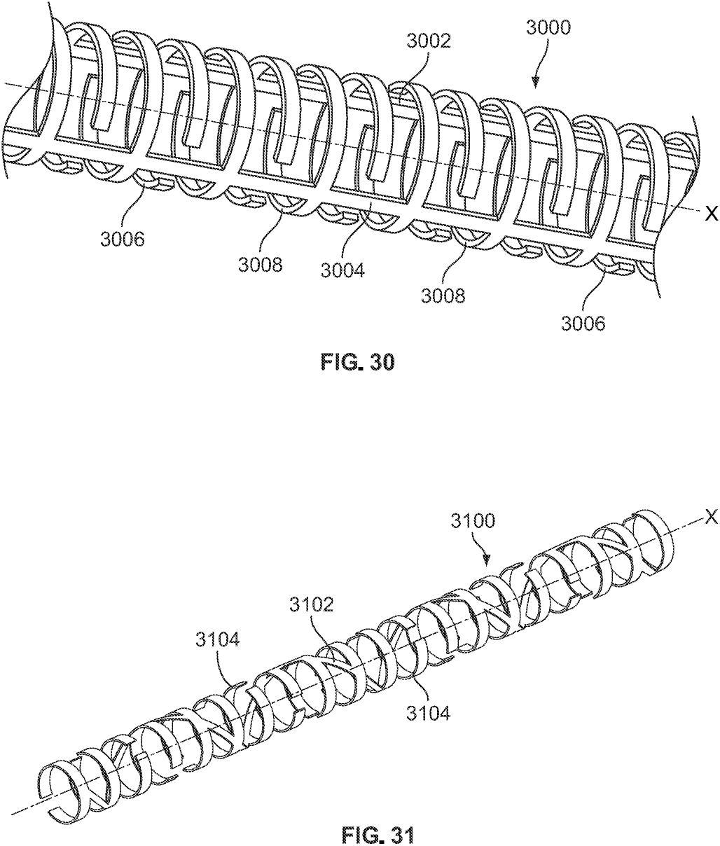

FIG. 31 shows an exemplary frame 3100 similar to frame 2800 of FIG. 28, but which includes a spiral-shaped spine. Thus, in the example of FIG. 31, frame 3100 also includes a spine 3102 which connects multiple split rings 3104 that are spaced apart along the length of the frame 3100. However, in this example, spine 3102 extend helically around longitudinal axis X. Here as well, each of split rings 3104 extends from the spine 3102 in a circumferential direction about longitudinal axis X, and may be spaced apart longitudinally by any suitable distance, which may be uniform or varied along the length of the frame 3100. In this example, as spine 3102 follows a helical path about longitudinal axis X, the gaps in each split ring 3104 likewise fall along a helical path about the longitudinal axis.

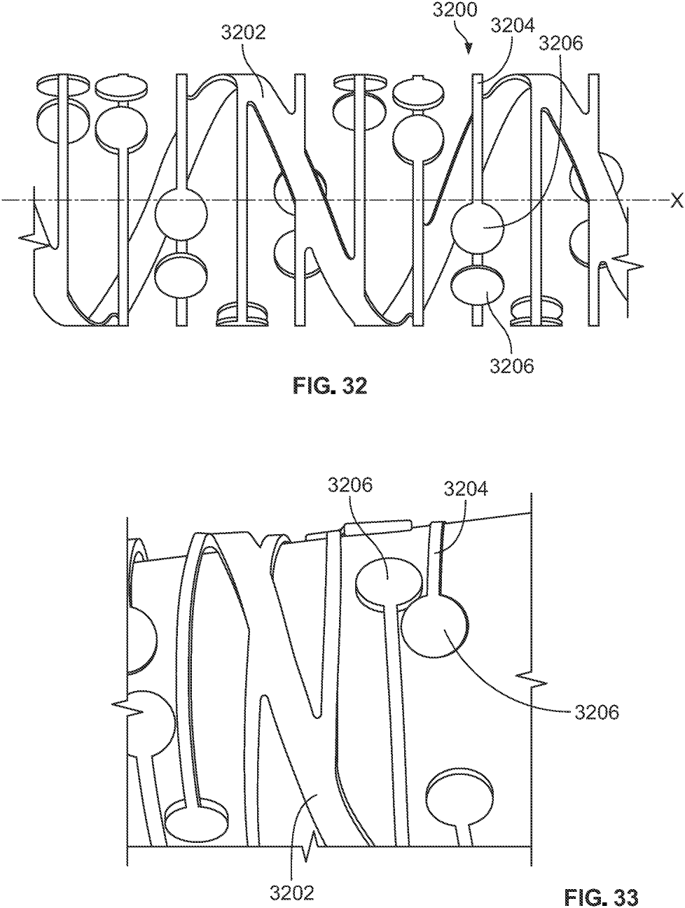

FIGS. 32 and 33 show an exemplary frame 3200 similar to frame 3100 of FIG. 31, but in which the ends of each split ring include lobes 3206. Like FIG. 31, frame 3200 of FIGS. 32 and 33 has a spiral-shaped spine 3202 which extends helically along longitudinal axis X (i.e., the length of the frame), and multiple split rings 3204 that are spaced apart along the length of the frame 3200. The ends of each split ring 3204 have lobes 3206. The size and shape of lobes 3206 may be configured to interfere with adjacent split rings 3204 when the frame 3200 is compressed, thus providing increased column strength under compression. In addition, lobes 3206 may provide a larger area for adhesion between the frame 3200 and other materials of the sheath body (e.g., a polymer or elastomer coating), and may help prevent the ends of each split ring 3204 from becoming caught on a medical device as it is passed through the sheath body.

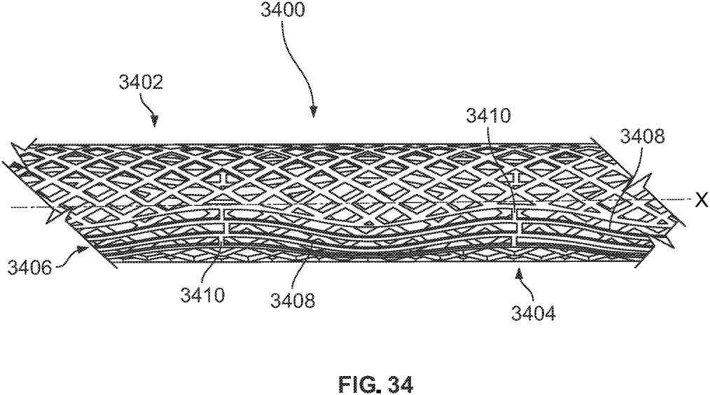

FIG. 34 shows an exemplary frame 3400 that includes multiple patterns arranged in bands that extend axially, not radially. In FIG. 34, a pair of a first axially extending band patterns 3402, 3404 (each extending along a longitudinal axis X) are separated by a pair of a second axially extending band patterns 3406 which each extend along the longitudinal axis X. In this example, the pair of first axial bands 3402, 3404 have a diamond pattern, and the pair of second axial bands 3406 include a plurality of wavy spines 3408 that extend along the longitudinal axis X and which are connected to one another via a plurality of connecting bridges 3410. The pair of first axial bands 3202, 3204 may be configured to enable a sufficient amount of longitudinal extension to give the frame 3400 adequate kink resistance during anticipated uses, while the second axial bands 3406 may be configured to enable a sufficient amount of radial expansion to permit the passage of larger objects through the frame 3400 (e.g., a medical device such as an intracardiac heart pump). Therefore, although second axial bands 3406 extend axially, they are radial expansion bands in terms of the expansion that they enable.

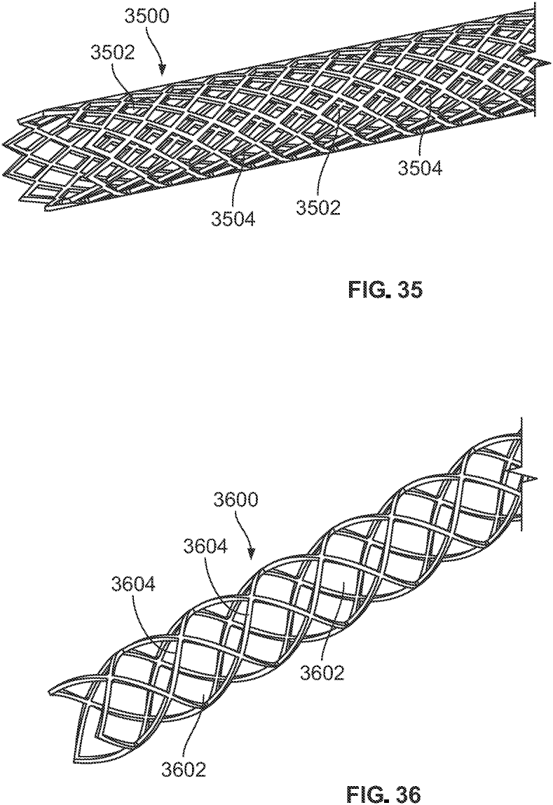

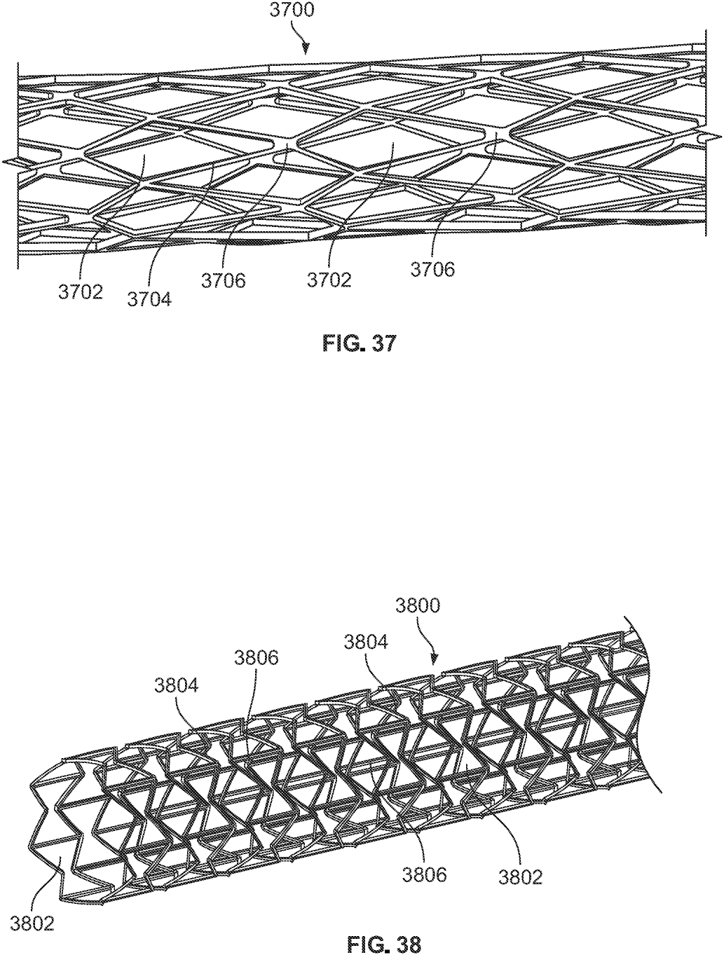

FIGS. 35-37 show various exemplary frames 3500, 3600, 3700 which each include a different diamond pattern that extends axially along the length of the frame. As will be appreciated, the size and shape of the diamond-shaped (i.e., a diamond-like pattern where apertures in the frame have a diamond-like but not precisely diamond shape) apertures 3502, 3602, 3702 may be configured to provide different amounts of radial expansion, kink resistance, and column strength. Any suitable shape of such apertures may be employed. Thus, as can be seen, the frames 3500, 3600 in FIGS. 35 and 36 are formed by a pair of helical ribs 3504, 3604 that extend the length of the frame 3500, 3600, and which intersect (because one extends clockwise and another extends counter clockwise) to define the pattern of diamond-shaped apertures 3502, 3602. Likewise, as can be seen in FIG. 37, the diamond-like shaped apertures 3702 of frame 3700 are asymmetrical, and thus are formed by stepped ribs 3704 which run the length of the frame 3700 and intersect at steps 3706.

FIG. 38 shows an exemplary frame 3800 that includes a plurality of bowtie-shaped apertures 3802 formed by connecting a plurality of zigzag radial expansion bands 3804 with a plurality of straight connecting bridges 3806. As will be appreciated, the bowtie-shaped pattern of the frame 3800 may be configured such that the frame 3800 will extend (as opposed to shorten) in the axial direction when it is exposed to a force causing it to expand radially (e.g., during insertion of a medical device such as an intracardiac heart pump), thus reducing the risk of finger-trapping.

In all examples described herein, the frame material and coating material may be selected to allow for thin frame walls while maintaining axial stiffness and elasticity. The coating may be made of a material such as a polymer. The polymer coating can be silicone or thermoplastic polyurethane. In some instances, the polymer fully may cover the entire length of the frame and the sheath body may exhibit a homogenous construction (frame and coating) along the entire length of the sheath. In other instances, the coating may extend over a proximal portion of the frame, covering between 5 and 50% in length of the proximal portion of the frame. Alternatively, the coating may extend over a distal portion of the frame, covering between 5 and 50% in length of the distal portion of the frame. In other instances, the coating may extend over any portion of the frame, and cover between 5 and 95% of the length of the frame. In other instances, the coating may extend over multiple portions of the frame, and the portions can be discontinuous in length, and/or discontinuous in circumference. The polymer encapsulation may be of a low elastic modulus as exhibited by typical Shore A Silicones and Shore A and Shore D thermoplastic polyurethanes. The material for coating the frame can be varied specific to the performance requirements of the expandable sheath body 202. A material with a lower elastic modulus may allow for lower radial strength to promote expansion while a material with a higher elastic modulus may allow for stronger durability to prevent coating failure during use. Elastomers that are urethane based may allow for additional hydrophilic coatings on the inner and outer layer to reduce frictional forces experienced by the opening and inner surface of the blood vessel. Thicker elastomer coatings may be beneficial for the durability of the coating and increase the stiffness of the expandable sheath body 202. Thinner elastomer coatings may promote radial expansion and allow for delivery of the heart pump through smaller sheath profiles. Materials may further be selected to be biocompatible such that they can be in direct and continuous contact with blood within the circulatory system for up to 28 days. Any of the materials described above can be used in any expandable sheath frame configuration, including for example configurations using any of the frame designs discussed above.

At least one advantage of a metallic and polymer/elastomer composite construction for the frame and coating of the sheath body may be that it allows a thin-walled construction ≤ 200 microns (0.008”), which may minimize arteriotomy size, improve vessel closure, and minimize vascular complications (i.e., bleeding/oozing). In contrast, conventional polymer sheaths capable of passing a 14Fr device may have wall thicknesses around ~400 micron and conventional sheaths capable of passing a 23Fr device may have wall thicknesses around ~680 micron. At least another advantage of a metallic and polymer/elastomer composite construction for the frame and coating may be that it allows the expandable sheath to retain sufficient column strength (e.g., for suitable pushability and/or buckling resistance) while maintaining sufficient bending flexibility and/or axial extendibility (e.g., for kink resistance), which may not otherwise be achievable with other thin-walled constructions.

In one aspect, described herein is an expandable sheath comprising a tubular frame extending along a longitudinal axis from a proximal end to distal end; the tubular frame comprising: a plurality of radial expansion bands, each radial expansion band at least partially, and optionally fully, extending around a circumference of the tubular frame, and a plurality of connecting bridges, wherein each connecting bridge couples a portion of one radial expansion band to a portion of an adjacent radial expansion band, wherein the radial expansion bands and connecting bridges are formed by laser cutting the tubular frame; and a coating formed over at least an exterior of the tubular frame.

According to any of the above aspects, the plurality of radial expansion bands and the plurality of connecting bridges alternate along one of the longitudinal axis of the frame or the circumference of the frame.

According to any of the above aspects, each radial expansion band comprises a plurality of band segments that define a zig-zag pattern around the circumference of the frame.

According to any of the above aspects, each radial expansion band extends around the circumference of the frame substantially perpendicularly to the longitudinal axis.

According to any of the above aspects, each radial expansion band extends around the circumference of the frame obliquely with respect to the longitudinal axis.

According to any of the above aspects, each radial expansion band comprises a continuous band strut that is continuous within the radial expansion band.

According to any of the above aspects, each radial expansion band includes at least two discontinuous portions, and wherein the discontinuous portions are connected by at least one connection bridge.

According to any of the above aspects, each connection bridge comprises portions configured to interfere with one another when the frame is exposed to a compressive force at least partially along the longitudinal axis.

From the foregoing and with reference to the various figures, those skilled in the art will appreciate that certain modifications can also be made to the present disclosure without departing from the scope of the same. While several aspects of the disclosure have been shown in the figures, it is not intended that the disclosure be limited thereto, as it is intended that the disclosure be as broad in scope as the art will allow and that the specification be read likewise. Therefore, the above description should not be construed as limiting, but merely as exemplifications of particular aspects of the present technology.

1. An expandable sheath comprising:
a tubular frame extending along a longitudinal axis from a proximal end to distal end, the tubular frame comprising:
a plurality of radial expansion bands, each radial expansion band at least partially extending around a circumference of the tubular frame, and
a plurality of connecting bridges, wherein each connecting bridge couples a portion of one radial expansion band to a portion of an adjacent radial expansion band, wherein the radial expansion bands and connecting bridges are formed by laser cutting the tubular frame; and
a coating formed over at least an exterior of the tubular frame.

2. The expandable sheath of claim 1, wherein the plurality of radial expansion bands and the plurality of connecting bridges alternate along one of the longitudinal axis of the tubular frame or the circumference of the frame.

3. The expandable sheath of claim 1, wherein each radial expansion band comprises a plurality of band segments that define a zig-zag pattern around the circumference of the tubular frame.

4. The expandable sheath of claim 1, wherein each radial expansion band extends around the circumference of the tubular frame substantially perpendicularly to the longitudinal axis.

5. The expandable sheath of claim 1, wherein each radial expansion band extends around the circumference of the tubular frame obliquely with respect to the longitudinal axis.

6. The expandable sheath of claim 1, wherein each radial expansion band comprises a continuous band strut that is continuous within the radial expansion band.

7. The expandable sheath of claim 1, wherein each radial expansion band includes at least two discontinuous portions, and wherein the discontinuous portions are connected by at least one connection bridge.

8. The expandable sheath of claim 1, wherein each connecting bridge comprises portions configured to interfere with one another when the tubular frame is exposed to a compressive force at least partially along the longitudinal axis.

9. The expandable sheath of claim 1, wherein each of the radial expansion bands extends around the entire circumference of the tubular frame.
